(19) United States
(12) Patent Application Publication
(10) Pub. No.: US 2015/0045576 A1
(43) Pub. Date: Feb. 12, 2015

(54) METHODS OF MAKING ALKYL LACTATES AND ALKYL LEVULINATES FROM SACCHARIDES

(71) Applicant: Battelle Memorial Institute, Columbus, OH (US)

(72) Inventors: Herman P. Benecke, Columbus, OH (US); Daniel Garbark, Blacklick, OH (US)

(21) Appl. No.: 14/453,426

(22) Filed: Aug. 6, 2014

Related U.S. Application Data
(60) Provisional application No. 61/863,776, filed on Aug. 8, 2013.

(51) Int. Cl. C07C 51/41; C07C 67/00; C07C 51/367

(52) U.S. Cl. CPC C07C 51/41; C07C 51/367; C07C 67/00
USPC 560/175; 562/515

(57) ABSTRACT

Unique methods have been developed to convert saccharides into value-added products such as alkyl lactates, lactic acid, alkyl levulinates, levulinic acid, and optionally alkyl formate esters and/or hydroxymethylfurfural (HMF). Useful catalysts include Lewis acid catalysts and Brønsted acid catalysts including mineral acids, metal halides, immobilized heterogeneous catalysts functionalized with a Brønsted acid group or a Lewis acid group, or combinations thereof. The saccharides are contacted with the catalyst in the presence of various alcohols.

### BACKGROUND OF THE INVENTION

Monosaccharides and polysaccharides such as fructose, glucose and sucrose are abundant materials that can potentially serve as sources of valuable commercial biobased products or intermediates to prepare such products. By biobased, we mean prepared from a plant or animal source. However, monosaccharides such as fructose and glucose are also food components, so conversion of these materials to industrial usage reduces the availability of these saccharides to serve as food stocks. Thus, there is a need to use non-edible saccharides for the production of industrial products.

Soy molasses is a high volume and non-edible byproduct obtained from the production of soy protein isolate and soy protein concentrates from soybeans. It is used mainly for animal feed. Soy molasses is typically produced as about a 50-60% solids soy molasses solution. Sugars constitute about 62 percent of these solids. Sucrose (a disaccharide composed of fructose and glucose), raffinose (a trisaccharide composed of fructose, glucose and galactose), and stachyose (a tetrasaccharide composed of the same monosaccharides) constitute about 96% of the sugar fraction the monosaccharides fructose and glucose comprise the rest. Saponins which constitute up to about 15% of the solid fraction also contribute saccharides to this composition. Currently, some producers ferment the soy molasses solution to produce methane that is used as a fuel.

Soy molasses could serve as a source for producing value-added biobased products which would provide more value than using it as a fuel.

EP 2184270 reports the conversion of glucose, fructose, xylose and sucrose to racemic methyl lactate in yields of 40-60% when these sugars are heated to 160° C. in the presence of methanol and relatively high concentrations of zeolite tin-beta. This zeolite has tin (IV) molecularly incorporated in its structure. The article “Tin-catalyzed conversion of trioses to alkyl lactates in alcohol solutions”, Chem. Commun. 2005, 2716-2718 (Y. Hayashi and Y. Sasaki) described the conversion of dihydroxyacetone (DHA) and glyceraldehyde to alkyl lactates with about 90% yield when heated to 90° C. with primary alcohols in the presence of tin(II) and tin(IV) chloride catalysts. The article “Zeolite H-USY for the production of lactic acid and methyl lactate from C3-sugars”, J. Catalysis 2010, 269, 122-130 (R. M. West, et al.) demonstrated that a zeolite with a low Si/A1 composition (Zeolyst-Y) was effective in catalyzing the reaction of DHA and glyceraldehyde with methanol to form methyl lactate when heated to 115° C. when using this zeolite catalyst at relatively high concentrations. However, this catalyst lost activity with continued use due to coking, which implies that it would have limited use in a commercial process for converting DHA to alkyl lactates.

The article “Catalytic conversion of cellulose to Levulinic acid by metal chlorides, Molecules 2010, 15, 5258-5272 (L. Peng, et al.) demonstrated that transition metal chlorides, especially chromium(III) chloride, and aluminum chloride exhibit catalytic activity in converting cellulose and glucose to levulinic acid in 55-65% yield when heated to 200° C. in water. A disadvantage of this process is that the levulinic acid is produced as a very dilute aqueous solution so the relatively high cost to evaporate water detracts from the economics of this process.

In “Dehydration of fructose in non-aqueous media”, D. W. Brown, et al, Chem. Tech. Biotechnol, 1982, 32, 920, fructose was heated in lower alcohols such as methanol in the presence of Amberlyst-15. A yield of 43% 5-methoxymethyl-2-furfural(methoxy HMF) and 47% methyl levulinate was obtained in methanol. The selectivity in making methyl levulinate was only about 42% (conversion×yield). Furthermore, when other products such as methoxy HMF are made, a fractionation scheme must be developed to remove the other products, which increases the cost of the process.

There is a need for economical processes of converting non-edible saccharides into useful industrial products.

### SUMMARY OF THE INVENTION

Processes have been developed to convert saccharides to at least one of alkyl lactates, lactic acid, alkyl levulinates, and levulinic acid.

One aspect of the invention is a method of making at least one of an alkyl lactate, lactic acid, an alkyl levulinate, and levulinic acid. In one embodiment, the method includes contacting a saccharide with a catalyst comprising a Lewis acid or a Brønsted acid in the presence of a polyol at a temperature in a range of about 100° C. to about 200° C. to form a reaction mixture comprising the at least one of the alkyl lactate, the lactic acid, the alkyl levulinate, and the levulinic acid.

In another embodiment, the method includes heating a polysaccharide to a temperature in a range of about 65° C. to about 85° C. in the presence of water and a first Brønsted acid to hydrolyze the polysaccharide into at least one monosaccharide. The monosaccharide is contacted with a catalyst comprising a Lewis acid or a Brønsted acid in the presence of an alcohol at a temperature in a range of about 100° C. to about 200° C. to form a reaction mixture comprising the at least one of the alkyl lactate, the lactic acid, the alkyl levulinate, and the levulinic acid.

In another embodiment, the method includes heating a polysaccharide containing an alpha(1→6) acetal linkage to a temperature in a range of about 130° C. to about 170° C. in the presence of a catalyst comprising a mineral acid or an immobilized heterogeneous catalyst functionalized with a Brønsted acid group or a Lewis acid group, water, and an alcohol to hydrolyze the polysaccharide into at least one monosaccharide that was bonded by the alpha(1→6) acetal linkage and to react the at least one monosaccharide to form a reaction mixture comprising the at least one of the alkyl lactate, the lactic acid, the alkyl levulinate, and the levulinic acid.

In still another embodiment, the method includes contacting an aqueous solution containing at least about 5% of at least one saccharide with a catalyst comprising a Lewis acid or a Brønsted acid in the presence of an alcohol at a temperature in a range of about 100° C. to about 200° C. to form a reaction mixture comprising the at least one of the alkyl lactate, the lactic acid, the alkyl levulinate, and the levulinic acid. The alcohol has a solubility in water of less than 10%, and an aqueous phase and a non-aqueous phase are formed. The non-aqueous phase includes the alcohol and the at least one of the alkyl lactate, the lactic acid, the alkyl levulinate, and the levulinic acid. The catalyst consists essentially of a mineral acid, a metal halide catalyst, an immobilized heterogeneous catalyst functionalized with a Brønsted acid group or a Lewis acid group, or combinations thereof.

### DETAILED DESCRIPTION OF THE INVENTION

Processes have been developed to convert saccharides to racemic lactic acid esters and levulinic acid esters, as well as racemic lactic acid and levulinic acid.

Lactic acid is listed by the Department of Energy (DOE) as one of the top-30 biobased intermediates to prepare food additives, detergents, biobased solvents as well as polylactic acid (which requires L-lactic acid). Almost all lactic acid used today is produced from the fermentation of glucose, which makes lactic acid a target of the “food for industrial use” controversy.

Levulinic acid is listed by the DOE as one of the top-10 biobased intermediates, and its uses include PVC plasticizers, lubricants, surfactants and solvents. Levulinic acid can also be readily converted to methyl tetrahydrofuran which is used as a gasoline oxygenate, while esters of levulinic acid are being increasingly used as diesel fuel oxygenates. The main levulinic acid production process involves heating cellulosic feedstocks to about 200-220° C. in the presence of sulfuric acid to produce levulinic acid and formic acid in yields of 50% and 20%, respectively with a 30% yield of char.

Although not wishing to be bound by theory, it is believed that levulinic acid and levulinic esters are formed by the acid catalyzed dehydration of monosaccharides in water and alcohols in a process that involves formation of the intermediate hydroxymethylfurfural (HMF). HMF then undergoes rehydration in converting to levulinic acid that is mainly esterified to levulinic esters in the presence of solvent alcohols.

While the formation of lactic acid and lactic esters is believed to proceed by a different mechanism (which involves a reverse aldol cleavage to C₃ components), some Lewis acid catalysts can generate both levulinic esters and lactic esters.

Humins are non-desired polymeric byproducts that are formed by the acid-catalyzed cross-reaction of non-reacted sugars with the initially formed HMF. Both soluble and insoluble humins are formed and their presence is readily indicated by their deep black color. It is desirable to minimize the amount of humins produced in the reaction.

The saccharides can be monosaccharides, disaccharides, trisaccharides, tetrasaccharides, or other higher polysaccharides, such as cellulose and starch. Suitable saccharides include, but are not limited to, fructose, glucose, sucrose, raffinose, stachyose, galactose, maltose, cellobiose, melibiose, cellulose, starch, other polysaccharides, or combinations thereof. The saccharides can be edible or non-edible.

In some embodiments, the saccharides are contacted with a catalyst comprising a Lewis acid or a Brønsted acid in the presence of an alcohol to form at least one of an alkyl lactate, lactic acid, an alkyl levulinate, and levulinic acid.

Suitable catalysts include, but are not limited to, soluble metal halide catalysts, solid immobilized heterogeneous catalysts functionalized with a Brønsted acid group or a Lewis acid group, or mineral acids. Suitable soluble metal halide catalysts include, but are not limited to, tin(II) halides, such as tin(II) chloride, tin(IV) halides, such as tin(IV) chloride, zinc(II) halides, such as zinc(II) chloride, and aluminum halides, such as aluminum(III) chloride, and combinations thereof. Zeolyst-Y (available from Zeolyst International) can be present with the soluble metal halide catalyst, if desired. Suitable immobilized heterogeneous catalysts functionalized with a Brønsted acid group or a Lewis acid group include, but are not limited to, at least one of metal halides, sulfonic acids, and sulfamic acids bound to various immobilized supports comprising at least one of silica gel, silica, an organic resin, and clay (i.e., metal halides bound to the various immobilized supports, sulfonic acids bound to the various immobilized supports, or sulfamic acids bound to the various immobilized supports). Suitable mineral acids include, but are not limited to, HCl, H₂SO₄, HNO₃, H₃PO₄, and combinations thereof.

The catalyst can be present in the range of about 0.01 to about 7 times the weight of the saccharide, or about 0.01 to about 6 times the weight of the saccharide, or about 0.01 to about 5 times the weight of the saccharide, or about 0.01 to about 4 times the weight of the saccharide, or about 0.01 to about 3.5 times the weight of the saccharide, or about 0.01 to about 2 times the weight of the saccharide, or about 0.01 to about 1 times the weight of the saccharide, or about 0.01 to about 0.75 times the weight of the saccharide, or about 0.01 to about 0.5 times the weight of the saccharide, or about 0.01 to about 0.25 times the weight of the saccharide, or about 0.01 to about 0.1 times the weight of the saccharide, or about 0.01 to about 0.05 times the weight of the saccharide.

The soluble metal catalyst can include one or more of tin(II) halides, tin(IV) halides, zinc(II) halides, or aluminum(III) halides. At high catalyst loadings, tin(II) halide can be present in an amount of about 0.2 to about 0.6 times the weight of the saccharide, tin(IV) halide can be present in an amount of about 0.2 to about 0.6 times the weight of the saccharide, and zinc(II) halide can be present in an amount about 20 to about 80% of a weight of the saccharide. The low catalyst loading is about 0.01 to about 0.2 times the weight of the saccharide, or about 0.01 to about 0.1 times the weight of saccharide. For example, tin(II) halide can be present in an amount of about 0.02 to about 0.04 times the weight of the saccharide, tin(IV) halide can be present in an amount of about 0.02 to about 0.06 times the weight of the saccharide, zinc(II) halide can be present in an amount about 0.02 to about 0.4 times a weight of the saccharide. The aluminum(III) halide can be present in the amounts indicated above for the tin and zinc halides.

Zeolyst-Y can be present with the soluble metal catalyst in an amount about 0.6 to about 0.8 times the weight of the saccharide. The presence of zeolyst-Y reduced product yields, so it is less desirable to include it. However, in some circumstances, it might be acceptable to include it.

The immobilized heterogeneous catalyst having a Brønsted acid group or a Lewis acid group can be present in an amount of 0.01 to about 7 times the weight of saccharide, or from about 0.01 to about 3.5 times the weight of saccharide, or about 0.01 to about 2 times the weight of the saccharide, or about 0.01 to about 1 times the weight of the saccharide, or about 0.01 to about 0.75 times the weight of the saccharide, or about 0.01 to about 0.5 times the weight of the saccharide, or about 0.01 to about 0.25 times the weight of the saccharide, or about 0.01 to about 0.1 times the weight of the saccharide, or about 0.01 to about 0.05 times the weight of the saccharide, or about 0.2 to about 1 times the weight of the saccharide.

The mineral acid can be present in an amount of about 0.1 M to about 4.0 M. The volume of the mineral acid can range from about 2 ml of mineral acid/g saccharide to about 20 ml of mineral acid/g saccharide.

The alcohol can be a mono-alcohol, a polyol, or combinations thereof. Suitable alcohols include, but are not limited to, methanol, ethanol, 1-propanol, 2-propanol, 1-butanol, 2-butanol, isobutyl alcohol, tert-butyl alcohol, 1-pentanol, 1-hexanol, or combinations thereof. Suitable polyols include, but are not limited to, ethylene glycol, 1,2-propylene glycol, 1,3-propylene glycol, 2-methyl-1,3-propylene glycol, butane-1,2-diol, or combinations thereof.

The alcohol can be present in an amount of about 2 ml/g saccharide to about 20 ml/g saccharide, or 7 ml/g to about 12 ml/g.

In some embodiments, the alcohol can be combined with water. The water can be present in an amount of about 3 to about 50% of the alcohol volume, or 8 to about 15% of the alcohol volume.

The saccharides are contacted with the catalyst at a temperature of at least about 100° C., or at least about 110° C., or at least about 120° C., or at least about 130° C., or in a range of about 100° C. to about 200° C., or about 110° C. to about 180° C.

The reaction time is generally at least about 3 hr, or at least about 5 hr, or at least about 6 hr, or at least about 8 hr, or at least about 10 hr, or in the range of about 3 hr to about 50 hr, or about 3 hr to about 40 hr, or about 3 hr to about 30 hr, or about 3 hr to about 24 hr, or about 3 hr to about 20 hr, or about 3 hr to about 15 hr, or about 3 hr to about 10 hr.

Brønsted acids have selectivities for levulinic esters over lactic esters of at least 50:1. The conversions of Brønsted acids with various saccharides range from about 35% to about 95%. Lewis acids generally favor lactic esters, but some favor levulinic esters with conversions ranging from about 40% to about 85%.

In some embodiments, the saccharide is hydrolyzed. This can involve a pre-hydrolysis step before the saccharide is contacted with the catalyst or an in situ hydrolysis step. In the pre-hydrolysis step, a polysaccharide is heated to a temperature in a range of about 65° C. to about 85° C., or about 70° C. to about 80° C. in the presence of water and a Brønsted acid to hydrolyze the polysaccharide into at least one monosaccharide. The monosaccharide is then contacted with the catalyst. Suitable Brønsted acids for the pre-hydrolysis step include, but are not limited to, sulfuric acid, hydrochloric acid, perchloric acid, nitric acid, aluminum chloride, immobilized Brønsted acids, including SiliaBond® propylsulfonic acid, and immobilized Lewis acids, including SiliaBond® aluminum chloride.

In the case of in situ hydrolysis, in some embodiments, the catalyst is an immobilized heterogeneous catalyst functionalized with a Brønsted acid group or a Lewis acid group, or a metal halide. The polysaccharide is hydrolyzed by heating the polysaccharide to a temperature in a range of about 110° C. to about 170° C., or about 110° C. to about 170° C., while contacting the saccharide with the catalyst. In some embodiments, the polysaccharide contains an alpha(1→6) acetal linkage typically between galactose and galactose or galactose and glucose. The polysaccharide is heated to a temperature in a range of about 155° C. to about 170° C. in the presence of an immobilized heterogeneous catalyst having a Brønsted acid group, or a Lewis acid group, or a mineral acid and an alcohol/water mixture to hydrolyze the polysaccharide. The polysaccharide is hydrolyzed and the alpha(1→6) acetal linkage is broken, At least one of the monosaccharides which was previously bonded by the alpha(1→6) acetal linkage reacts to form a reaction mixture comprising the at least one of the alkyl lactate, the lactic acid, the alkyl levulinate, and the levulinic acid.

In some embodiments, the process involves forming two phases. An aqueous solution of saccharides is contacted with an alcohol having a solubility in water of less than 10 wt %, or less than 5 wt %, or less than 2.5 wt %, or less than 1 wt %. Suitable alcohols include, but are not limited to, 1-butanol, 1-pentanol, 1-hexanol, or combinations thereof. The non-aqueous phase contains the alcohol and the alkyl lactate, the lactic acid, the alkyl levulinate, and/or the levulinic acid.

In some embodiments, the saccharide is an aqueous solution containing at least about 5 g saccharide/100 ml of aqueous solvent of at least one saccharide is used, or at least about 10 g saccharide/100 ml of aqueous solvent, or at least about 15 g saccharide/100 ml of aqueous solvent, or at least about 20 g saccharide/100 ml of aqueous solvent, or at least about 25 g saccharide/100 ml of aqueous solvent, or at least about 30 g saccharide/100 ml of aqueous solvent, or at least about 35 g saccharide/100 ml of aqueous solvent, or at least about 40 g saccharide/100 ml of aqueous solvent. The aqueous solution containing at least about 5 g saccharide/100 ml of aqueous solvent of at least one saccharide can be one or more of soy molasses, sorghum juice, beet juice, sugar cane, molasses derived from the purification of beet sugar, and molasses derived from the purification of sugar cane sugar. The catalyst comprises a Lewis acid or a Brønsted acid. Examples of suitable catalyst consist essentially of mineral acids, metal halide catalysts, and immobilized heterogeneous catalyst functionalized with a Brønsted acid group or a Lewis acid group, or combinations thereof.

There can be up to a total of 30 vol % of another alcohol, either a mono-alcohol or a polyol. The aqueous solution can be heated using microwave heating, or other suitable heating methods.

### Soluble Metal Halide Catalysts

Initial trials focused on the potential reaction of fructose with (−)-menthol or (S)-2-methyl-1-butanol in the presence of a catalyst package containing tin(II) chloride plus Zeolyst-Y plus zinc(II) chloride. These chiral alcohols were used with the intent of inducing asymmetric induction based on our earlier work in reacting DHA with these alcohols. However, no trace of the expected (−)-menthyl lactate or (S)-2-methyl-1-butyl lactate was detected in the reaction of fructose with these chiral alcohols using this catalyst package.

When this reaction was repeated using methanol rather than (−)-menthol or (S)-2-methyl-1-butanol while using this catalyst package, the formation of similar molar quantities of methyl lactate and methyl levulinate in a total yield of about 56 percent was observed. Detection and quantitation of these compounds was performed with ¹H NMR spectroscopy using p-xylene as an internal standard. The probable cause of the lack of reaction in (−)-menthol and (S)-2-methyl-1-butanol to form methyl lactate and methyl levulinate is that fructose is expected to be much more soluble in methanol than in these higher alcohols.

Methyl formate was also detected by NMR spectroscopy in almost equal molar quantities as methyl levulinate in most of the reaction mixtures. This observation strongly suggests that both methyl levulinate and methyl formate were formed from the production and cleavage of hydroxymethylfurfural (HMF) to form these two compounds. Co-production of methyl lactate and methyl levulinate/methyl formate from fructose suggests it underwent two fundamentally different type reactions to produce these two sets of products.

Production of methyl lactate from fructose is believed to proceed via an initial reverse aldol reaction to form DHA and glyceraldehyde which are initially transformed to pyruvaldehyde in a tin chloride catalyzed process. It is believed that pyruvaldehyde is converted to its hemiacetal by reaction with methanol, and this hemiacetal rearranges to methyl lactate in another tin chloride catalyzed process. The higher expected reactivity of methanol versus (−)-menthol and (S)-2-methyl-1-butanol in forming hemiacetals with pyruvaldehyde could also contribute to the lack of reactivity of these bulkier alcohols.

Production of methyl levulinate/methyl formate can occur via a dehydration reactions whereby fructose undergoes cyclization through a series of dehydration reactions to form HMF which undergoes ring opening by reaction with water (water is formed during HMF formation) to form both levulinic acid and formic acid via a postulated complex reaction mechanism (Mechanism of Levulinic Acid Formation. J. Horvat, et al., Tetrahedron Letters 1985, 26, 2111). These acids would be expected to undergo esterification with methanol to form methyl levulinate and methyl formate. Given the positive results in the initial trials, a series of screening experiments was performed while varying the following parameters: 1) Concentrations and ratios of various catalysts; 2) Ratio of catalysts to substrate; 3) Water content; 4) Reaction temperature; and 5) Reaction time.

Trials were performed at a relatively high catalyst loading and a much lower catalyst loading, as shown in Tables 1 and 2. Some of the results are presented in Tables 1 and 2. Table 1 illustrates the reactions of fructose and sucrose with methanol at relatively high catalyst concentrations, while Table 2 focuses on the reactions of these saccharides with methanol at relatively low catalyst concentrations. Conversion values of saccharides and relative yields of methyl lactate and methyl levulinate were determined by ¹H NMR spectroscopy. The Tables provide the relative weights of catalysts converted into the weight percentage that each catalyst was of the saccharide being studied. Experiments are designated by the laboratory report book page number used for each experiment and the conversion of saccharides to methyl lactate and methyl levulinate as well as the molar yields of methyl lactate and methyl levulinate are shown.

For the reactions performed at relatively high catalyst concentrations, the following comments can be made. The weight of tin(II) chloride used in these experiments was set close to 42 percent of the saccharide weight, and the weight of zinc(II) chloride was set close to 32 percent of the saccharide weight. Using these relative high quantities of the two catalysts and using specific ratios of catalysts and at specific temperatures, 100 percent and 79 percent combined yields of methyl lactate and methyl levulinate yields were obtained from fructose and sucrose, respectively. Separately omitting either tin(II) or zinc(II) chloride caused yield decreases, with the largest decrease occurring when tin(II) chloride was omitted. Since the total yields obtained when omitting either catalyst did not add up to the total yield obtained when these catalysts were used together, these comparative results suggest that both of these catalysts are active and operate in a synergistic manner. Reactions with sucrose without performing initial hydrolysis to monosaccharides also gave relatively high yields of methyl lactate and methyl levulinate. Small amounts of insoluble black material were observed in all reaction products and are assumed to be caramelized products from fructose or sucrose or humins or “char” from furanic compounds. Reaction times typically varied from 15-29 hours, and slightly decreased yields were observed when reactions were performed for extended periods of time.

TABLE 1
Reactions of Fructose and Sucrose with
Methanol at Relatively High Catalyst Loading

| | | | | | | |
|---|---|---|---|---|---|---|
| LRB 58707: | 5 | 15 | 16 | 21 | 22 | 17 |
| Saccharide: | Fructose | Fructose | Fructose | Fructose | Fructose | Sucrose |
| Zeolyst (% of | 71.9 | 0 | 0 | 0 | 0 | 0 |
| Sacch.): | | | | | | |
| SnCl₂ (% of | 42.0 | 42.3 | 42.4 | 42.1 | 0.0 | 41.9 |
| Sacch.): | | | | | | |
| ZnC1₂ (% of | 30.8 | 31.0 | 30.3 | 0.0 | 30.7 | 31.4 |
| Sacch.): | | | | | | |
| Temp. (° C.): | 160 | 160 | 130 | 130 | 130 | 130 |
| Mole % Me. | 28.9 | 39.3 | 53.2 | 42.0 | 14.5 | 38.7 |
| Lactate: | | | | | | |
| Mole % Me. | 21.9 | 22.9 | 39.2 | 17.2 | 2.0 | 32.4 |
| Levulinate: | | | | | | |
| Total Conversion | 50.9 | 62.3 | 92.5 | 59.2 | 16.5 | 71.1 |
| %: | | | | | | |

For the reactions performed at relatively low catalyst concentrations, the following comments can be made. For experiments using fructose, the total methyl lactate plus methyl levulinate yields ranged up to about 60 percent when tin(II) chloride and zinc(II) chloride at loadings of 2.6-5.9% of fructose concentrations were used. When fructose was the starting material, production of methyl levulinate was favored by tin(IV) chloride, while methyl lactate was favored by tin(II) chloride. For the limited experiments involving sucrose, the total yield of both esters was 59 percent with a tin(II) chloride and zinc chloride loading of 3.6% and 2.9%, respectively. Reactions with sucrose without performing initial hydrolysis to monosaccharides gave relatively high yields of methyl lactate and methyl levulinate. As mentioned above, insoluble black materials which are assumed to be caramelized sugars or humins or “char” were also observed in all reaction products.

TABLE 2
Reactions of Fructose and Sucrose with
Methanol at Relatively Low Catalyst Loading

| | | | | | |
|---|---|---|---|---|---|
| LRB 53707: | 23 | 25 | 28 | 26 | — |
| LRB 53989: | — | — | — | — | 23 |
| Saccharide: | Fructose | Fructose | Fructose | Fructose | Sucrose |
| SnCl₂ (% Sacch.): | 3.6 | 0 | 0 | 0 | 3.6 |
| SnCl₄ (% Sacch.): | 0 | 2.9 | 2.6 | 5.6 | 00 |
| ZnCl₂ (% Sacch.): | 3.0 | 3.4 | 3.1 | 3.0 | 2.9 |
| Temp. (° C.): | 130 | 130 | 130 | 130 | 150 |
| Mole % Me Lact. | | | | | |
| @ 10 hr: | 49.6 | 37.4 | 37.8 | 34.9 | 44.1 |
| @ 20 hr: | — | — | — | — | 51.7 |
| Mole % Me Lev. | | | | | |
| @ 10 hr: | 4.9 | 15.0 | 15.0 | 20.6 | 9.5 |
| @ 20 hr: | — | — | — | — | 11.6 |
| Total conversion | 54.7 | 52.5 | 52.8 | 55.5 | 53.6 @ 10 hr |
| at max. time: | | | | | 63.3 @ 20 hr |

Several conclusions can be made. A process was developed using tin and zinc chloride catalysts for converting monosaccharides and polysaccharides to methyl lactate and methyl levulinate in relatively high yields. The relative amounts of methyl lactate versus methyl levulinate were found to be somewhat adjustable based on the ratio of tin(II) to tin(IV) chloride and also based on the ratio of tin(II) chloride to zinc chloride. Adjusting these catalyst ratios can provide a way for an operator to adjust production towards either methyl lactate or methyl levulinate depending on the market demand for either product. Reactions with sucrose at both high and low catalyst concentrations gave relatively high concentrations of methyl lactate and methyl levulinate without performing initial hydrolysis to monosaccharides.

A disadvantage of using soluble metal halide catalysts such as tin(II) chloride, tin(IV) chloride and zinc chloride is that these catalysts are difficult to recover after the reactions are completed. Potential methods for catalyst recovery include, but are not limited to, cooling the reaction mixtures to low temperatures and filtering the reaction mixture, and/or adding solvents such as tetrahydrofuran (THF) or p-dioxane, which are soluble in alcohols but in which metal chloride catalysts have relatively low solubilities, or performing a combination of these two approaches, in order to precipitate these catalysts.

Immobilized Catalysts Functionalized with a Brønsted Acid Group or a Lewis Acid Group

Immobilized heterogeneous catalysts functionalized with a Brønsted acid group or a Lewis acid group are insoluble in the reaction solvent and reaction mixture in which they are employed. An advantage of these catalysts is that they can be removed efficiently from reaction mixtures by simple filtration. Removal of catalysts will allow their recycle in consecutively performed batch reactions. In flow-through reactors, immobilized catalysts can be fixed in the reactor so they will not be spread throughout the reactor effluent.

The following results were obtained from preparing an immobilized catalyst by reaction of silica gel with tin(IV) chloride in ethanol as described in [“Grafting SnCl4 catalyst as a novel solid acid for the synthesis of 3-methylbut-3-en-1-ol”, M. Ji, et al., Catalysis Today 173 (2011), 28-31]. This catalyst has been used to catalyze the Prins reaction which is very different reaction than the current reactions of saccharides. In this type of catalyst, the SnCl₃ species become attached to the silica gel particles through Si—O—SnCl₃ bonding to generate a catalyst with significant amount of Lewis type acidity. This material was used to catalyze the reaction of fructose in methanol at 140° C. for 14 hours to generate methyl lactate, methyl levulinate and HMF in the following yields: methyl lactate (60.9%), methyl levulinate (7.6%) and HMF (3.7%). A blank reaction was performed wherein fructose was heated in methanol with non-derivatized silica gel under the same reaction conditions to obtain methyl lactate in <1.7% yield, no methyl levulinate, and only a trace of HMF. These results indicate that the silica gel carrier had a minimal effect on the activity of this hybrid catalyst and that reacting silica gel with tin(IV) chloride was responsible for its catalytic effect.

Another reaction was performed with a commercial product SiliaBond® propylsulfonic acid which has propanesulfonic acid moieties bonded to silica particles and thus has Brønsted acid properties (available from SiliCycle, Inc. of Quebec, Canada). This material was used to catalyze the reaction of fructose in methanol at 120° C. for 24 hours to obtain only methyl levulinate in about a 86% yield which indicates that this is a very efficient process to convert fructose to an industrially viable product.

### Hydrolysis of Polysaccharides

Effective processes for hydrolysis of polysaccharides are needed because only monosaccharides can be converted to HMF.

One process involves the pre-hydrolysis of polysaccharides. The polysaccharides were heated in water in the presence of a Brønsted acid. Suitable Brønsted acids include, but are not limited to sulfuric acid, hydrochloric acid, phosphoric acid, nitric acid, and immobilized heterogeneous catalyst functionalized with a Brønsted acid group or a Lewis acid group.

Pre-hydrolysis of both sucrose and raffinose were obtained by heating these sugars in water in the presence of Brønsted acids at a temperature in the range of about 65° C. to about 85° C., or about 70° C. to about 80° C. Silica supported SiliaBond® propyl sulfonic acid (SPSA) available from SiliCycle Inc. of Quebec Canada) was used with sucrose, and nearly quantitative production of glucose and fructose was observed in one hour. It is believed that this temperature range initiates polysaccharide cleavage while minimally initiating HMF formation (since HMF would react further with non-reacted sugars to generate appreciable amounts of humins). When working with raffinose, 0.33M and 0.66M sulfuric acid were used, and the very rapid formation of fructose and the disaccharide melibiose, a disaccharide of glucose and galactose, was observed. Hydrolysis of melibiose occurred slowly while obtaining nearly complete hydrolysis in 15 hours when using 0.66M sulfuric acid. At this time, 88.5 mole percent of all monosaccharides had been released.

In these experiments, 1.5 g raffinose (to the nearest 0.1 mg) was placed in vial along with 6 mL of either 0.33M or 0.67M sulfuric acid and heated in a tube heater at 72-73° C. After various time intervals, the vials were removed for analysis by high performance liquid chromatography (HPLC) after previously determining the retention times of fructose, glucose, galactose (the monosaccharides composing raffinose) and melibiose, the disaccharide of glucose and galactose formed after the relatively labile acetal bond between fructose and glucose is cleaved. Response factors were determined for these saccharides but not for galactose due to the similarity of its retention time to that of glucose and its very low response factor. Using these response factors, the relative weight and mole percentages of fructose, glucose and melibiose were determined while assuming that the percentage of galactose was the same as glucose (since galactose and glucose are formed from the hydrolysis of melibiose). After 4 hours heating, raffinose was completely hydrolyzed at each sulfuric acid concentration and the remaining hydrolytic event involved the hydrolysis of melibiose. At 15 hours, the mole percent of remaining melibiose in the 0.67M sulfuric acid solutions was down to 6.1% which corresponded to 11.5 mole percent of total monosaccharide still bound in melibiose. Only a small amount of insoluble humins were present in this sample. By contrast, at 15 hours the mole percent of remaining melibiose in the 0.33M sulfuric acid solution was only down to 41.5% which corresponded to 58.6 mole percent of total monosaccharide still bound in melibiose. Thus, using the 0.67M sulfuric acid at 72-73° C. provides an efficient and convenient manner to achieve a high percent hydrolytic cleavage of hydrolytic-resistant polysaccharides incorporating the difficult-to-hydrolyze alpha(1-6) acetal linkages between glucose and galactose.

An in-situ hydrolysis process was also developed. Effective in-situ hydrolysis of raffinose to its constituent monosaccharides was obtained when using immobilized catalysts functionalized with Brønsted acid groups (e.g., SPSA) or Lewis acid groups (e.g., SiliaBond® aluminum chloride (SAC) available from SiliCycle Inc. of Quebec Canada) as well as with sulfuric acid in two-phase alcohol/aqueous phase systems.

The temperature ranges from about 110° C. to about 170° C., or about 130° C. to about 170° C. Fructose will react rapidly at about 110° C., but glucose needs a temperature about 30-40° C. higher to obtain similar reaction rates. At and above about 180° C., substantial decomposition takes place.

In-situ hydrolysis occurred during heating of the polysaccharide to 155-170° C. at which temperatures initially-generated monosaccharides are converted primarily to levulinates. The effectiveness of in-situ-hydrolysis was indicated by working with sucrose and raffinose wherein total levulinate yields were close to the theoretical yields expected from reaction of the individual monosaccharides (when examined individually, fructose and glucose gave about 95% yields while galactose gave about 50% yield).

When using SPSA, reaction of raffinose in methanol/water ((10:1) gave a 79% yield of levulinates plus a 10% yield of lactic ester and 1% yield of HMF (plus a currently non-determined small amount of acetone), while resulting in a 11.4% catalyst weight gain.

Brønsted Acid Catalysts.

Comparison of yield data from experiments 14 and 21 (Table 3) provides support for in-situ hydrolysis of raffinose where galactose and raffinose were heated in 100:10 methanol/water at 170 C in the presence of SiliaBond propyl sulfonic acid under identical reaction conditions. In experiment 14, galactose was shown to produce levulinates in 49 percent yield whereas in experiment 21, raffinose provided levulinates in 76 percent yield. If 100 percent in-situ hydrolysis was operative, and both fructose and glucose provided 100 percent levulinate yields, the expected total levulinate yield from raffinose would be 83 percent. Knowing from other experiments that fructose and glucose (experiment 49 in Table 3) provide yields in the mid-90 percent, these relative yields are consistent with raffinose undergoing nearly complete in-situ hydrolysis.

### Lewis Acid Catalysts.

Earlier work (Y. Yang, et al., Green Chem., 2012, 14, 509) showed that non-immobilized AlCl₃ in biphasic mixtures (water with NaCl/THF mixtures) apparently catalyzed in-situ hydrolysis of maltose, cellobiose, and cellulose, none of which contain the difficult-to-hydrolyze galactose alpha(1-6) acetal linkages.

Yield data from experiment 24 (Table 3) provides support that effective pre-hydrolysis of raffinose had occurred when raffinose was heated in 100:15 methanol/water at 170° C. in the presence of SiliaBond Aluminum Chloride since a 75 percent yield of levulinates were obtained while also obtaining yields of lactates and HMF in 10 and 1 percent yields (total yield of 86 percent). Thus, we have shown that an immobilized Lewis acid effectively catalyzes the alpha(1-6) acetal linkages in raffinose to generate its constituent monosaccharides

### Alcohols

The effects of using different alcohols on various saccharides were investigated.

With SPSA, the used catalysts were black. Scanning electron microscopy (SEM) indicated that humins had mainly adsorbed on the used catalyst particles because no different sized humin particles were observed. SEM also indicated no reduction in particle size which is an important property of immobilized catalysts since attrition of immobilized catalyst typically results in change of catalytic activity and can cause filtration membrane clogging which impedes recycling of used catalyst

The relative reactivity when using SPSA is glucose >>galactose and the relative amount of humin formation is galactose >>glucose as determined by obtaining a levulinate yield of 96% and a catalyst weight gain of 5.97% (experiment 49) when reacting glucose in 100:10 methanol/water at 170° C. while obtaining a levulinate yield of 49% and a catalyst weight gain of 77% (experiment 14) in the same solvent system and reaction conditions.

Recent work by Hu, et al. suggests that humin production is reduced by in-situ reaction of saccharide and HMF aldehyde groups with alcohols to generate acetal functionality that retards cross-reactions when using the immobilized Brønsted acid Amberlyst 70 in methanol/water [Xun Hu, et al., Bioresource Technology 102 (2011) 10104-10113]. Hu's data does not show the strong dependence of levulinate yields on methanol/water ratios that is demonstrated in the current application.

A 96% yield of levulinates and a 5.97% catalyst weight gain was obtained when reacting glucose at 170° C. in 90:10 methanol/water in the presence of SPSA (experiment 49), while a 36 percent levulinate yield and a 35.2% catalyst weight gain was obtained when glucose was reacted in 50:50 methanol/water under the same reaction conditions (Experiment 54). These results are consistent with the relative tendencies to convert the aldehyde groups of saccharides and HMF to acetals in methanol/water mixtures since acetal formation is an equilibrium reaction that produces water and thus is retarded by increased amounts of water.

In seeking to enhance acetalization effects, we evaluated ethylene glycol (EG) as an alternate reagent to increase levulinate yields and reduce humin formation.

In Experiment 11 (Table 3), the lowest catalyst weight uptake to-date (0.0160 g) was obtained when reacting glucose at 170° C. in 100% ethylene glycol (EG). Partial replacement of methanol in methanol/water solvent systems with EG (7%) led to increased levulinate yields in comparison. For comparison, galactose was converted to levulinates in 49% yield when reacting in 100:10 methanol/water in the presence of SPSA (Experiment 14 in Table 3) while the yield of levulinates increased to 58% in 93:7:10 methanol/EG/water (Experiment 15 in Table 3). Also, when raffinose was converted to levulinates in 100:10 methanol/water in the presence of SPSA the levulinate yield was 76% (Experiment 21 in Table 3) while the levulinate yield increased to 82% when 93:7:10 methanol/EG/water was used under the same reaction conditions (Experiment 16 in Table 3).

When reacting glucose at 170° C. in 100% EG, a very low catalyst weight increase of 0.88% was obtained. However, about 20 mole percent of a by-product was detected which is believed to be diethylene glycol. Levulinate yield increases and catalyst weight increases were also observed when partially replacing methanol with EG as demonstrated above. As previously mentioned, galactose was converted to levulinates in 49% yield with a corresponding 77% catalyst weight gain when reacting in 100:10 methanol/water in the presence of SPSA (Experiment 14 in Table 3). However, when galactose was reacted with 93:7:10 (methanol/EG/water), the levulinate yield increased to 58% and the catalyst weight gain was 19.5% (Experiment 15 in Table 3). Also when raffinose was converted to levulinates in 100:10 methanol/water in the presence of SPSA the levulinate yield was 77% and the catalyst weight gain was 9.48% (Experiment 21 in Table 3) while the levulinate yield increased to 82%, and the catalyst weight gain was 4.26% when 93:7:10 methanol/EG/water was used under the same reaction conditions (Experiment 16 in Table 3). No putative diethylene glycol was noted in these experiments incorporating partial replacement of methanol with EG.

### Two Phase Organic/Aqueous Phase Systems

Soy molasses is a heterogeneous aqueous suspension where the various sugars are fully solubilized, while other components such as crude proteins, peptides, lipids, phosphatides and ash are assumed to comprise at least part of the suspended material. Soy molasses resists filtration through membranes and does not separate well during centrifugation. The removal of the 50% water from the soy molasses either before or after reaction would be expensive. The use of an appropriate two phase organic/aqueous system where the water comes primarily from the soy molasses will keep many non-desired components of soy molasses, such as proteins and peptides, out of the organic phase while extracting the levulinate components into the organic phase.

Another industrial-scale water-rich saccharide solution is sorghum juice which contains about 18% sucrose. This system would also benefit from a two-phase system. Other water-rich saccharide mixtures include beet juice, sugar cane, and molasses that is derived from the purification of beet and sugar cane sugar.

V. E. Tarabanko, et al [Chemistry for Sustainable Development 13 (2005), 551-558] describe the use of C₄-C₈ monoalcohol/aqueous systems with sulfuric acid or sodium bisulfate as the Brønsted catalysts while reacting either dry fructose or sucrose. Using 1-butanol, they stated that when using sulfuric acid they obtained a single phase.

However, when raffinose or mixtures of fructose, glucose and galactose were reacted in 1-butanol/water systems, two phase systems were obtained both before initiating the reaction and after the reaction was completed. Tarabanko got better results using sodium bisulfate in 1-butanol that generated a two system. Tarabanko apparently only used sodium bisulfate with C₅-C₈ alcohols (see Table 3 of Tarabanko) and obtained low levulinate yields.

In the present application, good levulinate yields were obtained with threetwo-phase systems using various Brønsted acids with 1-butanol, 1-pentanol, and 1-hexanol. For example, HCl was used with 1-butanol (Experiment 53), SPSA was used with 1-pentanol (Experiment 35), and H₂SO₄ was used with 1-hexanol (Experiments 42, 44, 45, and 51).

In addition, Tarabanko did not demonstrate pre-hydrolysis or in-situ hydrolysis of difficult-to-hydrolyze polysaccharides such as raffinose and stachyose with any monoalcohol in either monophasic or biphasic conditions.

There is one significant advantage when using two-phase alcohol/aqueous solvent systems that Tarabanko did not obtain. This advantage is directly related to the composition of soy molasses. Soy molasses is currently prepared as a 60% solids/40% water mixture and about 60% of the solids are comprised of mono-, di-, tri- and tetrasaccharides. If one were to use a single phase solvent that would dissolve these sugars, the 40% water would remain in these solvents, and the cost of distilling this quantity of water would be quite high. Also, in a single phase system the product levulinates would be generated along with the 40% other compounds and impurities which would require significant amount of fractionation. By utilizing alcohols with less than 10% solubility in water, high yields compared to the Tarabanko system can be obtained.

The organic phases evaluated include 1-butanol, (7.3% soluble in water), 1-pentanol (2.2% solubility in water) and 1-hexanol (0.59% solubility in water) (which also implies low solubilities of water in these alcohols). The work focused on 1-hexanol where the products in the organic layer were hexyl levulinate and levulinic acid.

Trials were initially performed with raffinose, 1-hexanol and 0.40M HCl (no pre-hydrolysis), and the following results were obtained by analyzing the contents of the 1-hexanol layer. When raffinose was reacted using 0.40M hydrochloric acid at 170° C., a 62 percent yield of levulinates was obtained, and the amount of separated humins amounted to 3.0% of the starting raffinose mass (experiment 36 in Table 3). However, in this reaction appreciable amounts of 1-chlorohexane and dihexyl ether were identified. It is believed that the 1-chlorohexane originated from the initial chlorination of 1-hexanol which then reacted further with 1-hexanol to form dihexyl ether. Subsequently, this reaction was repeated, but at 155° C. to obtain a 58 percent yield of levulinates with less 1-chlorohexane and dihexyl ether, and the amount of separated humins was 16.90% of the starting raffinose mass (experiment 37 in Table 3).

In one set of experiments, the reaction of raffinose was compared to a reaction using equivalent amounts of fructose, glucose and galactose under identical reaction conditions and almost identical levulinate yields were obtained. In experiment 42 in Table 3, raffinose was reacted with 1.68M sulfuric acid at 155° C. to obtain levulinates in 56 percent yield. This experiment was repeated, except that raffinose was replaced with equivalent amounts of fructose, glucose and galactose, to obtain a levulinate yield of 56% and the separated humins constituted 7.08% of the starting saccharide mass (experiment 44 in Table 3). These results provide concrete evidence for the in-situ hydrolysis of raffinose and the difficult-to-hydrolyze galactose alpha(1-6) acetal linkages in this and similar two-phase alcohol/water solvent systems.

In experiment 51 in Table 3, a reaction was performed with fructose, glucose and galactose that was identical to experiment 44 except that 11% of the 1-hexanol was substituted with ethylene glycol (EG); the levulinate yield was 59 percent and the isolated humins were 5.07% of the starting saccharide mass. Comparison of these results from experiments 51 and 44 demonstrates the effectiveness of EG in increasing levulinate yields and decreasing humin formation in two-phase alcohol/aqueous phase systems.

Experiment 55 in Table 3 describes the reaction of 60% soy molasses at 155° C. using 1-hexanol and 2.02M sulfuric acid that when mixed with the water provided by soy molasses constituted the aqueous phase and diluting the effective molarity of sulfuric acid to 1.43M. A levulinate yield of 56 percent was obtained in the 1-hexanol layer which was clean and essentially devoid of non-desired water and the many impurities present in soy molasses including humins. It was noted that appreciable emulsification had occurred in the reaction mixture; this effect is ascribed to saponins which are known surfactants.

Due to the well known effect of salts in dispersing emulsions, the observed emulsification could be controlled by adding salts such as phosphates, nitrates, or sulfates. Suitable salts include, but are not limited to, sodium phosphate, sodium hydrogen phosphate, sodium dihydrogen phosphate, sodium sulfate, sodium bisulfate, or sodium nitrate, or combinations thereof. Alternatively, commercially available defoaming materials could be used to advantage.

In some embodiments, saponins could be removed from the aqueous solution containing the saccharide. Suitable methods for removing the saponins include, but are not limited to, solvent extraction or pre-hydrolysis. Solvent extraction will involve using water-insoluble solvents that best match the solubility parameters of the saponins. Solubility parameters can be calculated, and the results of these calculations would be used to down-select solvent candidates.

Small amounts of polyols can be used along with the alcohols which have a solubility in water of less than about 10%. The concentrations could range from 5% to about 30% so as to maintain effective two-phase behavior. Monoalcohols could also be use in concentrations of 10% to about 30% so as to maintain effective two-phase behavior.

In summary, the use of water insoluble alcohols with soy molasses (or other aqueous saccharide solutions) provides an efficient method that does not require distillation of significant amounts of water from soy molasses and also to devise purification schemes to remove the many impurities present in soy molasses. By contrast, if water soluble alcohols were used, the water and numerous impurities would be contained in the reaction mixture that would require removal and the incorporation of purification schemes.

TABLE 3
Reactions Performed in 300 mL Autoclave at 600 RPM

| Rx. | | Organic | | | p- | Rx. | Rx. | Product Analysis | | | | |
|---|---|---|---|---|---|---|---|---|---|---|---|---|
| Mixture | Sacch. | Solvent | H2O | Cat. | Xylene | Temp | Time | % | Mole % | Mole % | | |
| 54258- | (g) | (ml) | (ml) | (g) | (g) | (° C.) | (hr) | Yield | Me. lev. | Lev. Ac. | Other | Comments |
| 11-18 | Glucose | EG | 10 | SPSA¹ | 3.9946 | 170 | 5 | | | | | Cat wt. gain: |
| 11-23 | 8.0018 | (100) | | 1.8000 | | | 10 | | | | | 0.0159 g (0.88%) |
| 14-15 | Galactose | MeOH | 10 | SPSA¹ | 4.0026 | 170 | 8 | ~44 | | | | Cat. wt. gain: |
| 14-20 | 8.0016 | (100) | | 1.8006 | | | 20 | 49.4 | | | | 0.77 g (77%) |
| 15-15 | Galactose | MeOH | 10 | SPSA¹ | 4.0087 | 170 | 5 | 49.6 | | | | Cat. wt. gain: |
| 15-19 | 8.0040 | (93); | | 1.8021 | | | 10 | 57.6 | | | | 0.3523 (19.5%) |
| | | EG (7) | | | | | | | | | | |
| 16-17 | Raffinose | MeOH | 10 | SPSA¹ | 4.0052 | 170 | 5 | 66.2 | | | | Cat. wt. gain: |
| 16-22 | 7.9999 | (93); | | 1.7996 | | | 10 | 82.1 | | | | 0.0770 (4.26%) |
| | | EG (7) | | | | | | | | | | |
| 21-14 | Raffinose | MeOH | 10 | SPSA¹ | 4.0009 | 170 | 5 | 61.6 | | | | Cat. wt. gain: |
| 21-18 | 8.0019 | (100) | | 1.7995 | | | 10 | 75.9 | | | | 0.1706 (9.48%) |
| | | | | 22.5% of | | | | | | | | |
| | | | | raffinose | | | | | | | | |
| 24-15 | Raffinose | MeOH | 18.8 | SAC² | 4.4995 | 170 | 5 | 76.2 | | | LA: 10.1% | Cat. wt. gain: |
| 24-18 | 9.9996 | (125) | (13.1%) | 4.4995 | (45.0% | | 10 | 79.2 | 74.6 | 25.4 | HMF: 1.0% | 0.5124 g (11.42% |
| | | | | 45.0% of | of raffi- | | | | | | Acetone: small | wt. gain). |
| | | | | raffinose | nose) | | | | | | | |
| 24-21 | | | | | | | 15 | 78.9 | | | | |
| 34-14 | Raffinose | 1-hexanol | 70 | SPSA¹ | 4.0004 | 170 | 5 | 29.9 | | | HMF: 6.1% | Cat. wt. gain: |
| 34-17 | 8.0018 | (100) | | 1.8005 | | | 10 | 39.4 | | | HMF: 4.4% | 0.2615 g |
| 34-20 | | | | | | | 15 | 42.4 | 64.8 | 35.2 | HMF: 3.1% | |
| 35-14 | Raffinose | 1-pentanol | 70 | SPSA¹ | 3.9958 | 170 | 5 | 29.6 | 59.8 | 40.2 | HMF: 11.7% | |
| 35-17 | 7.9954 | (100) | | 1.7982 | | | 10 | 37.2 | | | HMF: 8.5% | |
| 36-14 | Raffinose | 1-hexanol | 70 | 0.40M | 4.0031 | 170 | 5 | 57.0 | | | HMF: 0.7% | Filtered solids: |
| 36-17 | 8.0009 | (100) | | HCl | | | 10 | 61.5 | 68.5 | 31.5 | | 0.2436 g |
| | | | | 70 mL | | | | | | | | (3.05%). |
| 37-13 | Raffinose | 1-hexanol | 70 | 0.40M | 4.0006 | 155 | 5 | 57.8 | | | HMF: 2.3% | Filtered solids: |
| 37-15 | 8.0013 | (100) | | HCl | | | 10 | 56.9 | 70.2 | 29.8 | HMF: 1.2% | 1.3522 g |
| | | | | 70 mL | | | | | | | | (16.9%). |
| 42-14 | Raffinose | 1-hexanol | 10 | 2.02M | 3.9961 | 155 | 5 | 56.4 | 78.1 | 21.9 | | Filtered solids: |
| 42-15 | 15.0004 | (90) | | H2SO4 | | | | | | | | 0.8823 g |
| | | | | 50 mL | | | | | | | | (5.88%). |
| | | | | 1.68M | | | | | | | | |
| 44-16 | Fructose | 1-hexanol | 10 | 2.02M | 4.0023 | 155 | 5 | 56.3 | 74.7 | 25.3 | | Filtered solids: |
| 44-19 | 4.5460 | (90) | | H2SO4 | | | 10 | 56.5 | | | | 0.9665 g |
| | Glucose | | | 50 mL | | | | | | | | (7.08%) |
| | 4.5454 | | | 1.68M | | | | | | | | |
| | Galactose | | | | | | | | | | | |
| | 4.5262 | | | | | | | | | | | |
| 45-16 | Fructose | 1-hexanol | 10 | 2.02M | 3.9953 | 170 | 5 | 55.7 | | | | Filtered solids: |
| | 4.5454 | (90) | | H2SO4 | | | 7.5 | | | | | 0.9872 g |
| | Glucose | | | 50 mL | | | | | | | | (7.23%) |
| | 4.5461 | | | 1.68M | | | | | | | | |
| | Galactose | | | | | | | | | | | |
| | 4.5457 | | | | | | | | | | | |
| 49-14 | Glucose | MeOH | 10 | 1.7999 | 3.9958 | 170 | 5 | 48.9 | | | HMF: 3.4% | Filtered solids: |
| 49-17 | 8.0001 | (100) | | SPSA¹ | | | 10 | 96.4 | | | HMF: 2.69% | 0.1074 g |
| | | | | | | | | | | | | (5.97%) |
| 51-17 | Fructose | 1-hexanol | 10 | 2.02M | 3.9952 | 155 | 5 | 58.9 | | | | Filtered solids: |
| 51-19 | 4.5454 | (81) | | H2SO4 | | | 10 | 57.8 | | | | 0.6925 g |
| | Glucose | EG (9) | | 50 mL | | | | | | | | (5.07%) |
| | 4.5458 | | | 1.68M | | | | | | | | |
| | Galactose | | | | | | | | | | | |
| | 4.5460 | | | | | | | | | | | |
| 53-17 | Raffinose | 1-butanol | 35 | 0.80M | 3.9989 | 155 | 5 | 54.1 | | | | Filtered solids: |
| 53-19 | 8.0028 | (100) | | HCl | | | 10 | 54.6 | 63.7 | 36.3 | | 0.2898 g |
| | | | | 35 mL | | | | | | | | (3.62%) |
| | | | | 0.40M. | | | | | | | | |
| 54-13 | Glucose | Methanol | 50 | SPSA¹ | None | 170 | 5 | 27.1 | | | | Due to |
| 54-16 | 8.0017 | (50) | | 1.8009 | | | 10 | 36.0 | 42.9 | 57.1 | | insolubility of p- |
| | | | | | | | | | | | | xylene in 50:50 |
| | | | | | | | | | | | | methanol/water, |
| | | | | | | | | | | | | p-xylene was |
| | | | | | | | | | | | | weighed into |
| | | | | | | | | | | | | weighed amounts |
| | | | | | | | | | | | | of reaction products |
| | | | | | | | | | | | | in NMR tube. Catalyst |
| | | | | | | | | | | | | wt. gain: 0.9762 g |
| | | | | | | | | | | | | (35.2%). |
| 55-13 | Soy | 1-hexanol | None | 2.02M | 3.9988 | 155 | 5 | 54.8 | | | | |
| 55-16 | Molasses | (90) | | H2SO4 | | | 10 | 56.1 | 75.0 | 25.0 | | |
| | (48.39) | | | 60 mL | | | | | | | | |
| | | | | 1.43M | | | | | | | | |

(1) SPSA: SiliaBond ® Propylsulfonic acid
(2) SAC: SiliaBond ® Aluminum Chloride

By the term “about,” we mean within 10% of the value, or within 5%, or within 1%.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method of making at least one of an alkyl lactate, lactic acid, an alkyl levulinate, and levulinic acid comprising:
contacting a saccharide with a catalyst comprising a Lewis acid or a Brønsted acid in the presence of a polyol at a temperature in a range of about 100° C. to about 200° C. to form a reaction mixture comprising the at least one of the alkyl lactate, the lactic acid, the alkyl levulinate, and the levulinic acid.

2. The method of claim 1 wherein the polyol comprises ethylene glycol, 1,2-propylene glycol, 1,3-propylene glycol, 2-methyl-1,3-propylene glycol, butane-1,2-diol, or combinations thereof.

3. The method of claim 1 wherein contacting the saccharide with the catalyst takes place in the presence of the polyol, a mono-alcohol and water.

4. The method of claim 1 wherein the alcohol comprises methanol, ethanol, 1-propanol, 2-propanol, 1-butanol, 2-butanol, isobutyl alcohol, tert-butyl alcohol, 1-pentanol, 1-hexanol, or combinations thereof.

5. The method of claim 1 wherein the catalyst comprises a mineral acid, a metal halide catalyst, an immobilized heterogeneous catalyst functionalized with a Brønsted acid group or a Lewis acid group, or combinations thereof.

6. The method of claim 5 wherein the catalyst is the metal halide catalyst, and wherein the metal halide catalyst comprises at least one of tin(II) chloride, tin(IV) chloride, zinc(II) chloride, and aluminum(III) chloride.

7. The method of claim 5 wherein the catalyst is the immobilized heterogeneous catalyst functionalized with the Brønsted acid group or the Lewis acid group, and wherein the immobilized heterogeneous catalyst functionalized with the Brønsted acid group or the Lewis acid group comprises at least one of a metal halide, a sulfonic acid, and a sulfamic acid bound to an immobilized support comprising at least one of silica gel, silica, an organic resin, and clay.

8. The method of claim 5 wherein the catalyst is the mineral acid, and wherein the mineral acid comprises HCl, H₂SO₄, HNO₃, H₃PO₄, or combinations thereof.

9. The method of claim 1 wherein the saccharide is fructose, glucose, sucrose, raffinose, stachyose, galactose, maltose, cellobiose, mellibiose, cellulose, starch, or combinations thereof.

10. The method of claim 1 further comprising:
recovering the catalyst; and
recycling the catalyst.

11. The method of claim 1 wherein the saccharide is a polysaccharide and further comprising hydrolyzing the polysaccharide.

12. The method of claim 11 wherein the polysaccharide is hydrolyzed by heating the polysaccharide in the presence of water and a Brønsted acid to a temperature in a range of of about 65° C. to about 85° C. before contacting the saccharide with the catalyst.

13. The method of claim 11 wherein the catalyst is an immobilized heterogeneous catalyst functionalized with a Brønsted acid group or a Lewis acid group, or a metal halide, and wherein the polysaccharide is hydrolyzed in situ by heating the polysaccharide to a temperature in a range of about 110° C. to about 170° C. while contacting the saccharide with the catalyst.

14. A method of making at least one of an alkyl lactate, lactic acid, an alkyl levulinate, and levulinic acid comprising:
heating a polysaccharide to a temperature in a range of about 65° C. to about 85° C. in the presence of water and a first Brønsted acid to hydrolyze the polysaccharide into at least one monosaccharide;
contacting the at least one monosaccharide with a catalyst comprising a Lewis acid or a Brønsted acid in the presence of an alcohol at a temperature in a range of about 100° C. to about 200° C. to form a reaction mixture comprising the at least one of the alkyl lactate, the lactic acid, the alkyl levulinate, and the levulinic acid.

15. The method of claim 14 wherein the first Brønsted acid comprises H₂SO₄, HCl, H₃PO₄, HNO₃ immobilized heterogeneous catalyst functionalized with a Brønsted acid group or a Lewis acid group, or combinations thereof.

16. The method of claim 14 wherein the catalyst comprises a mineral acid, a metal halide catalyst, an immobilized heterogeneous catalyst functionalized with a Brønsted acid group or a Lewis acid group, or combinations thereof.

17. A method of making at least one of an alkyl lactate, lactic acid, an alkyl levulinate, and levulinic acid comprising:
heating a polysaccharide containing an alpha(1→6) acetal linkage to a temperature in a range of about 130° C. to about 170° C. in the presence of a catalyst comprising a mineral acid or an immobilized heterogeneous catalyst functionalized with a Brønsted acid group or a Lewis acid group, water, and an alcohol to hydrolyze the polysaccharide into at least one monosaccharide that was bonded by the alpha(1→6) acetal linkage and to react the at least one monosaccharide to form a reaction mixture comprising the at least one of the alkyl lactate, the lactic acid, the alkyl levulinate, and the levulinic acid.

18. The method of claim 17 wherein the an immobilized heterogeneous catalyst functionalized with the Brønsted acid group or the Lewis acid group comprises at least one of a metal halide, a sulfonic acid, or a sulfamic acid bound to an immobilized support comprising at least one of silica gel, silica, an organic resin, and clay.

19. A method of making at least one of an alkyl lactate, lactic acid, an alkyl levulinate, and levulinic acid comprising:
contacting an aqueous solution containing at least about 5 g saccharide/100 ml of aqueous solvent of at least one saccharide with a catalyst comprising a Lewis acid or a Brønsted acid in the presence of an alcohol at a temperature in a range of about 100° C. to about 200° C. to form a reaction mixture comprising the at least one of the alkyl lactate, the lactic acid, the alkyl levulinate, and the levulinic acid, wherein the alcohol has a solubility in water of less than 10%, wherein an aqueous phase and a non-aqueous phase are formed, the non-aqueous phase comprising the alcohol and the at least one of the alkyl lactate, the lactic acid, the alkyl levulinate, and the levulinic acid, and wherein the catalyst consists essentially of a mineral acid, a metal halide catalyst, an immobilized heterogeneous catalyst functionalized with a Brønsted acid group or a Lewis acid group, or combinations thereof.

20. The method of claim 19 wherein the alcohol comprises 1-pentanol, 1-hexanol, 1-butanol or combinations thereof.

21. The method of claim 19 further comprising:
removing saponins from the aqueous solution containing at least about 5 g saccharide/100 ml of aqueous solvent of the at least one saccharide by solvent extraction or pre-hydrolysis.

22. The method of claim 19 further comprising:
adding a salt to the the aqueous solution containing at least about 5 g saccharide/100 ml of aqueous solvent of the at least one saccharide to reduce emulsification of the aqueous solution containing at least about 5 g saccharide/100 ml of aqueous solvent of the at least one saccharide.

23. The method of claim 19 wherein contacting the aqueous solution containing at least about 5 g saccharide/100 ml of aqueous solvent of the at least one saccharide with the catalyst comprising the Lewis acid or the Brønsted acid in the presence of the alcohol comprises contacting the aqueous solution containing at least about 5 g saccharide/100 ml of aqueous solvent of the at least one saccharide with the catalyst comprising the Lewis acid or the Brønsted acid in the presence of a mono-alcohol and a polyol.

24. The method of claim 19 further comprising:
heating the aqueous solution containing at least about 5 g saccharide/100 ml of aqueous solvent of the at least one saccharide with the catalyst comprising the Lewis acid or the Brønsted acid in the presence of the alcohol using microwave heating to the temperature in the range of about 100° C. to about 200° C.

25. The method of claim 19 wherein the the aqueous solution containing at least about 5 g saccharide/100 ml of aqueous solvent of the at least one saccharide comprises at least one of soy molasses, sorghum juice, beet juice, sugar cane, molasses derived from the purification of beet sugar, and molasses derived from the purification of sugar cane sugar.
